(19) United States
(12) Patent Application Publication
(10) Pub. No.: US 2026/0299277 A1
(43) Pub. Date: Oct. 1, 2026

(54) ADJUSTABLE BINOCULAR TUBE AND SURGICAL MICROSCOPE

(71) Applicant: ZUMAX MEDICAL CO., LTD., Jiangsu (CN)

(72) Inventors: Jilong WANG, Jiangsu (CN); Jiuqi XU, Jiangsu (CN); Sheng GUAN, Jiangsu (CN); Tao QIU, Jiangsu (CN); Weizhong ZHOU, Jiangsu (CN)

(21) Appl. No.: 18/996,354

(22) PCT Filed: Jul. 29, 2022
(86) PCT No.: PCT/CN2022/108854

§ 371 Date: Jan. 17, 2025

(51) Int. Cl. G02B 21/20; A61B 90/25; G02B 21/00

(52) U.S. Cl. CPC G02B 21/20; A61B 90/25; G02B 21/0012

(57) ABSTRACT

An adjustable binocular tube includes a connecting base assembly, an interpupillary distance adjusting base, an eyepiece tube holder, and an eyepiece tube assembly, the interpupillary distance adjusting base is connected to the connecting base assembly, the eyepiece tube holder is connected to the interpupillary distance adjusting base, the eyepiece tube assembly is connected to the eyepiece tube holder, an optical hole is formed in the connecting base assembly, the eyepiece tube assembly is connected to the eyepiece tube holder by means of an adjusting mechanism, which includes an adjusting ring, the adjusting ring is connected to the eyepiece tube holder, and the adjusting ring is movable relative to the eyepiece tube holder in the axial direction of the adjusting ring, and the eyepiece tube assembly is connected to the adjusting ring; the connecting base assembly is provided with an optical lens capable of cutting into or cutting out the optical hole.

### CROSS-REFERENCE TO RELATED APPLICATIONS

This application claims priority to PCT/CN2022/108854, filed on Jul. 29, 2022, which is hereby incorporated by reference in its entirety.

### TECHNICAL FIELD

The present disclosure belongs to the field of medical apparatus and instruments, and relates to an adjustable binocular tube and a surgical microscope.

### BACKGROUND

At present, the application of surgical microscopes is becoming increasingly widespread, not only in complex clinical procedures, but also in many treatment procedures such as restorative dentistry, endodontics, periodontal disease, and microsurgery. Due to ergonomic reasons, the position and size of surgical microscopes should be adjusted according to the operator's correct working position to achieve optimal muscle and visual working conditions.

The main lens portion of a surgical microscope generally comprises bens body, a binocular tube, eyepieces, a zoom objective lens, and a hand shank, etc., in most surgeries, the lens of the surgical microscope should be positioned horizontally while the doctor can observe through the binocular tube. The basic principle of the binocular tube is to use a prism system to divide the image formed by the objective lens into left and right parts, and then observe them separately with two eyepieces, the difference in magnification between the two lens tubes should not be greater than 2%~2.5%, the images formed by the two lens tubes should be consistent in direction, the relative tilt should be no more than 20″~30″, the divergence angle of the two optical axes in the horizontal plane should be ≤40″, the convergence angle of the two optical axes in the horizontal plane should be ≤20″, and the brightness and field of view size of the images formed by the two lens tubes should be consistent.

Long term use of surgical microscopes causing muscle pain and tension is a common phenomenon among doctors in various departments during work, 78% of neurosurgeons report pain after one day of surgery, of which 83% suffer from musculoskeletal pain. Long term surgery combined with high mental concentration places a heavy burden on the doctor's body, such as the muscles in the head, neck, and upper back having to compensate for every inch of forward head movement to support an additional 10 pounds of weight, these compromised positions can easily cause pain and spread to the arms or areas, leading to headaches, chronic pain, and exhaustion.

The length of the eyepieces on the binocular tube of the existing surgical microscope is short and cannot be adjusted, making it difficult to adapt to different surgical positions of doctors, increasing muscle strain on the waist, neck, and shoulders, which is not in line with ergonomic design and affects surgical efficiency.

### SUMMARY

The present disclosure provides an adjustable binocular tube and a surgical microscope.

A first aspect provided an adjustable binocular tube, comprises a connecting base assembly, an interpupillary distance adjusting base, an eyepiece tube holder, and an eyepiece tube assembly, the interpupillary distance adjusting base is connected to the connecting base assembly, the eyepiece tube holder is connected to the interpupillary distance adjusting base, the eyepiece tube assembly is connected to the eyepiece tube holder, and an optical hole is formed in the connecting base assembly,
the eyepiece tube assembly is connected to the eyepiece tube holder by means of an adjusting mechanism, the adjusting mechanism comprises an adjusting ring, the adjusting ring is connected to the eyepiece tube holder, and the adjusting ring is movable relative to the eyepiece tube holder in the axial direction of the adjusting ring, and the eyepiece tube assembly is connected to the adjusting ring; the connecting base assembly is provided with an optical lens capable of cutting into or cutting out the optical hole.

In the technical solution mentioned above, the binocular tube has a first use state and a second use state, and in the first use state, the adjusting ring moves to a first position setting in a direction away from the eyepiece tube holder, and the optical lens is cut into the optical hole; in the second use state, the adjusting ring moves to a second position setting in a direction towards the eyepiece tube holder, and the optical lens is cut out the optical hole, that is, the binocular tube can achieve two levels of adjustment;
the binocular tube has a third use state and a fourth use state, in the third use state, the optical lens is cut out the optical hole, and the adjusting ring moves to a first position setting in a direction away from the eyepiece tube holder; in the fourth use state, the optical lens is cut into the optical hole, and the adjusting ring moves to a second position setting in a direction towards the eyepiece tube holder.

In one embodiment, one end of the adjusting ring is inserted into the interior of the eyepiece tube holder from one end of the eyepiece tube holder, and the adjusting ring is movable to extend out of the eyepiece tube holder or retract into the eyepiece tube holder; the eyepiece tube assembly is connected to the other end of the adjusting ring.

In one embodiment the eyepiece tube assembly is inserted into the other end of the adjusting ring, and a sleeve body is provided between the eyepiece tube assembly and the adjusting ring, and in one embodiment the sleeve body is a copper sleeve, which facilitates the connection of the eyepiece tube assembly, such as plug-in connection, etc.

In one embodiment, the adjusting mechanism further comprises a first limit ring connected to one end of the eyepiece tube holder, when in the first position setting, the adjusting ring moves to extend from the eyepiece tube holder until one end of the adjusting ring is in contact with the first limit ring, the first limit ring can, on the one hand, limit the detachment between the adjusting ring and the eyepiece tube holder, and on the other hand, provide a prompt for the adjusting ring to move to the first position setting to prevent exceeding the adjustment range.

In one embodiment, the adjusting mechanism further comprises a second limit ring fixedly sleeved on the other end of the adjusting ring, when in the second position setting, the adjusting ring moves to retract into the eyepiece tube holder until the second limit ring is in contact with the first limit ring, the second limit ring can, on the one hand, provide a prompt for the adjusting ring to move to the second position setting, and on the other hand, move the adjusting ring by operating the second limit ring.

In a technical solution mentioned above, the adjusting ring is threadedly connected to the eyepiece tube holder, for example, an external thread is provided on the periphery of one end of the adjusting ring, an internal thread is provided on the inner circumference of the eyepiece tube holder, and the relative position adjustment between the two is achieved by rotating the adjusting ring.

In a technical solution mentioned above, one of the adjusting ring and the eyepiece tube holder is provided with a sliding groove, and the other is provided with a sliding block, the sliding groove extends in the axial direction of the adjusting ring, and the sliding block is located inside the sliding groove, for example, the adjusting ring is provided with the sliding groove, and the eyepiece tube holder is provided with the sliding block.

In a technical solution mentioned above, the optical lens is arranged on the connecting base assembly via a switching mechanism, the switching mechanism comprises an adjusting block flexibly connected to the connecting base assembly and provided with a mounting hole and an avoidance hole, the optical lens is arranged on the mounting hole, and when the mounting hole is coaxial with the optical hole, the optical lens is cut into the optical hole; when the avoidance hole is coaxial with the optical hole, the optical lens is cut out the optical hole.

In one embodiment, the switching mechanism further comprises an elastic sheet, one end of the elastic sheet is fixed, and the other end forms a bent portion; the adjusting block is provided with a first positioning groove and a second positioning groove, when the optical lens is cut into the optical hole, the bent portion of the elastic sheet is clipped into the first positioning groove of the adjusting block; when the optical lens is cut out the optical hole, the bent portion of the elastic sheet is clipped into the second positioning groove of the adjusting block, which can determine whether the optical lens is switched in place by determining the elastic sheet is clipped into the first or second positioning groove.

In one embodiment the adjusting block is movably connected to the connecting base assembly.

In one embodiment, the switching mechanism further comprises an operating piece connected to the connecting base assembly and cooperating with the adjusting block to control the movement of the adjusting block; the operating piece is threadedly connected to the adjusting block.

In one embodiment, the switching mechanism further comprises a movement guiding assembly comprising a guide holder and a guide block, the guide holder is connected to the connecting base assembly, the guide block is connected to the guide holder and located on at least one side of the adjusting block, a guide channel is formed between the guide holder and the guide block and extends in the direction of movement of the adjusting block, the adjusting block is located within the guide channel, which can ensure the stable movement of the adjusting block through the movement guiding assembly.

In one embodiment, the adjusting block is movable in an up-down direction relative to the connecting base assembly, and the mounting hole and the avoidance hole are arranged in the up-down direction.

In a technical solution mentioned above, the optical lens is a single lens with negative optical power or a single lens with positive optical power.

In a technical solution mentioned above, the binocular tube further comprises an optical lens assembly, the optical lens assembly comprises a first right-angle prism, a first lens group, an isosceles right-angle prism, a second right-angle prism, a Porro prism, a second lens group and a third lens group distributed in sequence along the optical path direction, where the first lens group, the second lens group and the third lens group meet the following parameters:
the focal length f_{G2} of the first lens group satisfies 100 mm<f_{G2}<300 mm, the first lens group comprises a single lens with positive focal power and a doublet lens group with negative focal power, and the doublet lens group satisfies:

$$\frac{\vert {R}_{2} \vert}{{φ}_{2 / 2}} < 15 ,$$

where R₂ is the curvature radius of the cemented surface, and φ₂ is the effective pore size of the cemented surface;
the focal length f_{G3} of the second lens group satisfies −1<f_{G3}/f_{G2}<−0.2, the second lens group comprises a doublet lens group with negative focal power, and the doublet lens group satisfies:

$$\frac{\vert {R}_{3} \vert}{{φ}_{3 / 2}} < 10 ,$$

where R₃ is the curvature radius of the cemented surface, and φ₃ is the effective pore size of the cemented surface;
the focal length f_{G4} of the third lens group satisfies 1<f_{G2}/f_{G4}<10, the third lens group comprises a doublet lens group with positive focal power.

In one embodiment, the first right-angle prism, the first lens group, the isosceles right-angle prism and the second right-angle prism are arranged within the connecting base assembly; the Porro prism is arranged within the interpupillary distance adjusting base, and the second lens group and the third lens group are arranged within the eyepiece tube holder.

In a technical solution mentioned above, the connecting base assembly comprises a lower connecting base and an upper connecting base, the lower connecting base and the upper connecting base are rotatably connected to each other, the optical lens is arranged on the lower connecting base, and the interpupillary distance adjusting base is connected to the upper connecting base.

In a technical solution mentioned above, the eyepiece tube assembly comprises an eyepiece tube, an eyepiece adjusting tube, a diopter ring, a diopter adjusting ring and an eyepiece diaphragm, the eyepiece tube is connected to the adjusting ring, the eyepiece adjusting tube is connected to the eyepiece tube, the diopter ring is connected to the eyepiece adjusting tube through the diopter adjusting ring, and the eyepiece diaphragm is connected to the eyepiece tube.

A second aspect provides a surgical microscope having the above-mentioned adjustable binocular tube.

Due to the use of the above technical solutions, the present disclosure has the following advantages over the conventional art:

In the present disclosure, by means of the adjustment of the binocular tube, a doctor can use a surgical microscope in a comfortable posture, so that the doctor can focus on high-precision work all the time; the use requirements of different doctors are met, and working in an ergonomic posture facilitates reduction of fatigue during use of a surgical microscope.

### BRIEF DESCRIPTION OF THE DRAWINGS

FIG. 1 is a schematic stereoscopic diagram of a binocular tube in an embodiment;

FIG. 2 is a schematic side view of the binocular tube in an embodiment;

FIG. 3 is a schematic sectional view of an implementation of an adjustment mechanism in an embodiment (in a first position setting);

FIG. 4 is a schematic sectional view of the implementation of the adjustment mechanism in an embodiment (in a second position setting);

FIG. 5 is a schematic bottom view of the implementation of the adjustment mechanism in an embodiment;

FIG. 6 is a schematic sectional view of another implementation of the adjustment mechanism in an embodiment (in a first position setting);

FIG. 7 is a schematic sectional view of the another implementation of the adjustment mechanism in an embodiment (in a second position setting);

FIG. 8 is a schematic diagram of an extension distance of the binocular tube in a first use state in an embodiment;

FIG. 9 is a schematic diagram of the extension distance of the binocular tube in a second use state in an embodiment;

FIG. 10 is a schematic diagram of a binocular tube in the conventional art;

FIG. 11 is a schematic diagram of cut-in of an optical lens in an embodiment;

FIG. 12 is a schematic diagram of cut-out of an optical lens in an embodiment;

FIG. 13 is a schematic diagram of an optical lens assembly in first use state of the binocular tube in an embodiment;

FIG. 14 is a schematic diagram of an optical lens assembly in second use state of the binocular tube in an embodiment;

FIG. 15 is a schematic diagram of the radius and thickness parameters of the optical lens assembly in an embodiment;

FIG. 16 is a schematic diagram of the use state of the binocular tube in first use state in an embodiment;

FIG. 17 is a schematic diagram of the use state of the binocular tube in second use state in an embodiment.

In the above accompanying drawings:
10, lower connecting base; 100, optical hole; 11, upper connecting base;
2, interpupillary distance adjusting base;
3, eyepiece tube holder; 30, sliding block;
4, optical lens;
50, eyepiece tube; 51, eyepiece adjusting tube; 52, diopter ring; 53, diopter adjusting ring; 54, eyepiece diaphragm;
60, adjusting ring; 600, sliding groove; 61, first limit ring; 62, second limit ring; 63, sleeve body;
70, adjusting block; 700, mounting hole; 701, avoidance hole; 702, mounting block; 703, first positioning groove; 704, second positioning groove; 71, elastic sheet; 710, bent portion; 72, operating piece; 73, guide holder; 74, guide block;
80, first right-angle prism; 81, first lens group; 82, isosceles right-angle prism; 83, second right-angle prism; 84, Porro prism; 85, second lens group; 86, third lens group.
a, datum plane; b, contrast plane.

### DETAILED DESCRIPTION OF THE EMBODIMENTS

The technical solutions of the present disclosure will be described clearly and completely below with reference to the accompanying drawings. Apparently, the described embodiments are merely some of rather than all of the embodiments of the present disclosure. All other embodiments obtained by those of ordinary skill in the art without creative efforts based on the embodiments of the present disclosure shall fall within the protective scope of the present disclosure.

In the description of the present disclosure, it should be noted that the orientations or positional relationships indicated by the terms “center”, “upper”, “lower”, “left”, “right”, “vertical”, “horizontal”, “inner”, “outer”, etc. are based on those shown in the accompanying drawings, are only for the convenience of describing the present disclosure and simplifying the description, rather than indicating or implying that the indicated device(s) or element(s) must have a specific orientation, be constructed and operated in a specific orientation, and therefore should not be construed as a limitation of the present disclosure. Furthermore, the terms “first”, “second”, “third” and are used for descriptive purposes only and should not be construed to indicate or imply relative importance.

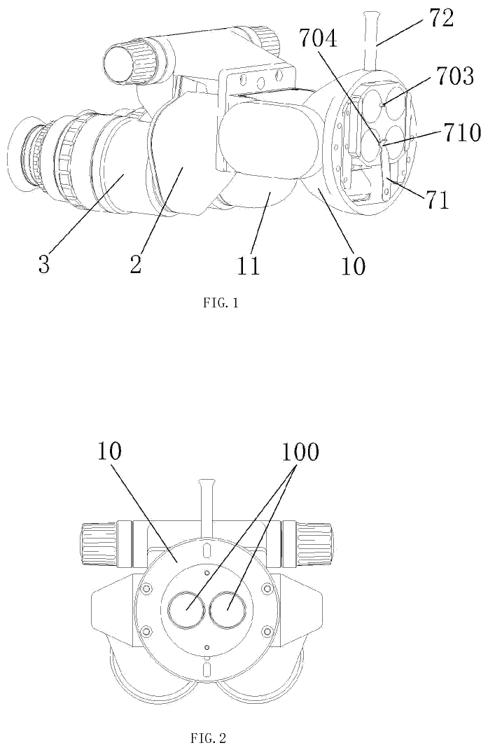

An adjustable binocular tube as shown in FIGS. 1 and 2, is suitable for use on surgical microscopes, and comprises a connecting base assembly, an interpupillary distance adjusting base 2, an eyepiece tube holder 3, an eyepiece tube assembly and an optical lens 4.

The connecting base assembly is used to connect with the body of a surgical microscope, and comprises a lower connecting base 10 and an upper connecting base 11, and the lower connecting base 10 and the upper connecting base 11 are rotatably connected to each other. Specifically, an end face of the lower connecting base 10 is a connecting end, which is connected to the body of the surgical microscope and is provided with an optical hole 100 to receive light from the body of the surgical microscope, the optical lens 4 is connected to the lower connecting base 10 and cooperates with the optical hole 100; the interpupillary distance adjusting base 2 is fixedly connected to upper connecting base 11.

The eyepiece tube holder 3 is fixedly connected to the interpupillary distance adjusting base 2, and the eyepiece tube assembly is connected to the eyepiece tube holder 3. The eyepiece tube assembly comprises an eyepiece tube 50, an eyepiece adjusting tube 51, a diopter ring 52, a diopter adjusting ring 53 and an eyepiece diaphragm 54, the eyepiece adjusting tube 51 is fixedly connected to the eyepiece tube 50, the diopter ring 52 is connected to the eyepiece adjusting tube 51 through the diopter adjusting ring 53, and the eyepiece diaphragm 54 is threadedly connected to the eyepiece tube 50. The eyepiece tube assembly has not been improved and will not be further described here.

In this embodiment, the eyepiece tube assembly is connected to the eyepiece tube holder 3 through an adjusting mechanism; the optical lens 4 is arranged on the end face of the lower connecting base 10 through a switching mechanism. The binocular tube has two use states in one implementation: the first use state and the second use state, through the cooperation of the adjusting mechanism and the switching mechanism, achieving two length adjustments of the binocular tube, as shown in FIGS. 16 and 17. The following provides a detailed description of the adjusting mechanism and the switching mechanism.

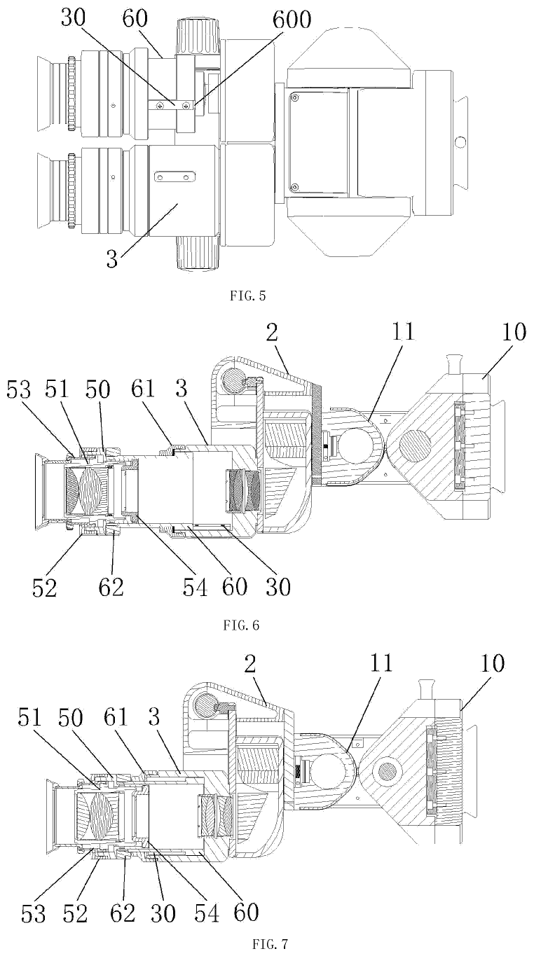

As shown in FIGS. 1-7, the adjusting mechanism comprises an adjusting ring 60, a first limit ring 61 and a second limit ring 62. Wherein, the adjusting ring 60 is connected to the eyepiece tube holder 3, and can move relative to the eyepiece tube holder 3 along the axis of the adjusting ring 60, and the eyepiece tube 50 of the eyepiece tube assembly is connected to the adjusting ring 60. Specifically, one end of the adjusting ring 60 is inserted into the interior of the eyepiece tube holder 3, and the adjusting ring 60 is movable to extend out of the eyepiece tube holder 3 or retract into the eyepiece tube holder 3, and the eyepiece tube 50 is inserted into the other end of the adjusting ring 60 by plugging and unplugging. A sleeve body 63 is provided between the eyepiece tube 50 and the adjusting ring 60, and in one embodiment the sleeve body 63 is a copper sleeve, which facilitates the plug-in connection of the eyepiece tube 50 to the adjusting ring 60. The first limit ring 61 is fixedly connected to one end of the eyepiece tube holder 3, and the second limit ring 62 is fixedly sleeved on the other end of the adjusting ring, which can achieve the movement of the adjusting ring 60 by operating the second limit ring 62.

When the binocular tube is in its first use state, the adjusting ring 60 moves to a first position setting in a direction away from the eyepiece tube holder 3, the first position setting is the position where the adjusting ring 60 extends out of the eyepiece tube holder 3 until one end of the adjusting ring 60 is in contact with the first limit ring 61; when the binocular tube is in the second use state, the adjusting ring 60 moves to a second position setting in a direction towards the eyepiece tube holder 3, the second position setting is the position where the adjusting ring 60 moves and retracts into the eyepiece tube holder 3 until the second limit ring 62 is in contact with the first limit ring 61.

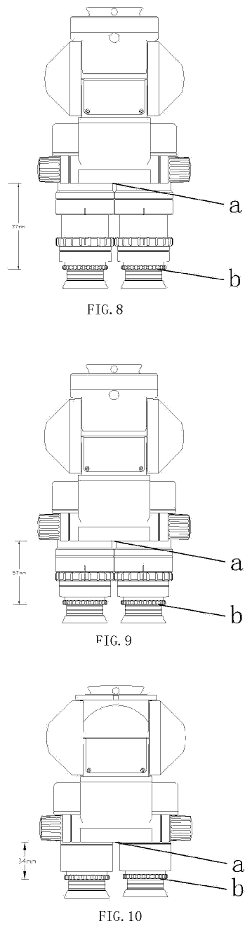

As shown in FIGS. 8-10, the length of the adjusting ring 60 is 20 mm, and in the first use state, the binocular tube can be extended by 23 mm compared to the existing structure, and in the second use state, the binocular tube can be extended by 43 mm compared to the existing structure. Of course, the extension length of the binocular tube can be adjusted according to the length of the adjusting ring 60.

Two implementation methods are given for the relative movement between the adjusting ring 60 and the eyepiece tube holder 3:

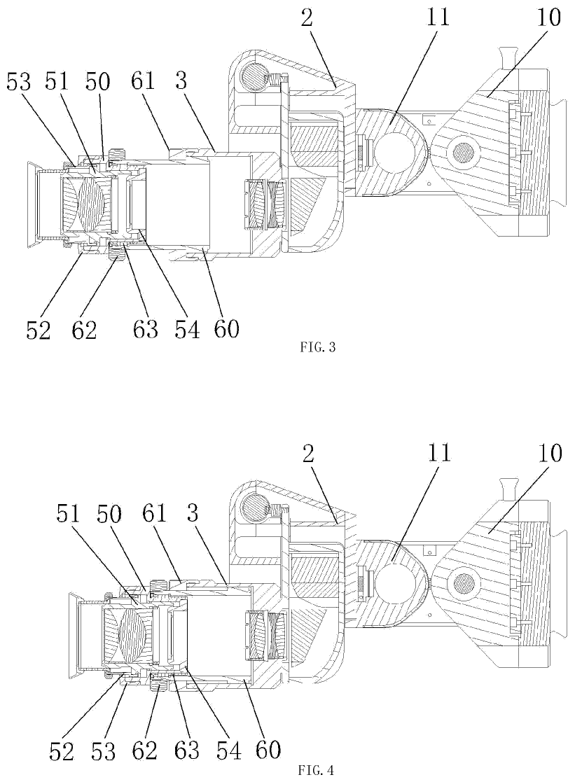

In one implementation, as shown in FIGS. 3 and 4, the adjusting ring 60 is threadedly connected to the eyepiece tube holder 3, specifically, an external thread is provided on the periphery of one end of the adjusting ring 60, an internal thread is provided on the inner circumference of the eyepiece tube holder 3, and the relative position adjustment between the two is achieved by rotating the adjusting ring 60.

In another implementation, as shown in FIGS. 5-7, one of the adjusting ring 60 and the eyepiece tube holder 3 is provided with a sliding groove 600, and the other is provided with a sliding block 30, the sliding groove 600 extends in the axial direction of the adjusting ring 60, and the sliding block 30 is located inside the sliding groove 600, specifically, the adjusting ring 60 is provided with a sliding groove 600, and the eyepiece tube holder 3 is provided with a sliding block 30.

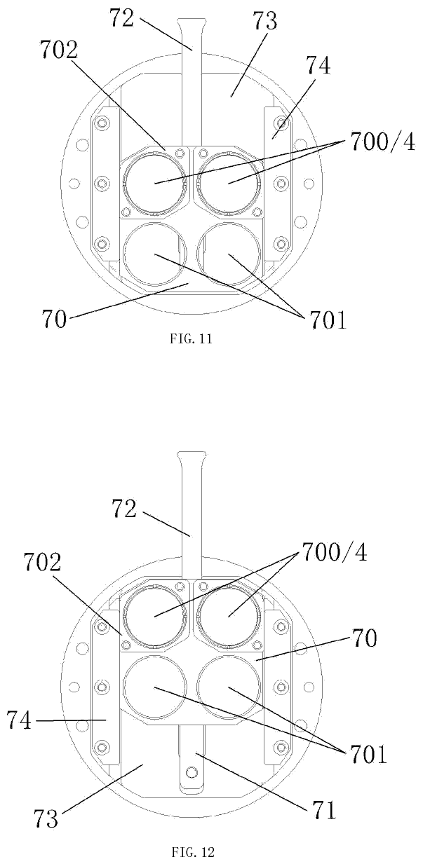

As shown in FIGS. 11 and 12, the switching mechanism comprises an adjusting block 70, an elastic sheet 71, an operating piece 72, and a movement guiding assembly. Wherein,

The adjusting block 70 is flexibly connected to the lower connecting base 10, specifically, the adjusting block 70 is movable up and down and is connected to the lower connecting base 10, the adjusting block 70 is provided with a mounting hole 700 and an avoidance hole 701, the mounting hole 700 and the avoidance hole 701 are provided in an up-down direction, a mounting block 702 is provided at the mounting hole 700, and the optical lens 4 is covered on the mounting hole 700 through the mounting block 702.

As shown in FIG. 11, when the binocular tube is in the first use state, the mounting hole 700 is coaxial with the optical hole 100, and the optical lens 4 on the mounting hole 700 is cut into the optical hole 100; as shown in FIG. 12, when the binocular tube is in the second use state, the avoidance hole 701 is coaxial with the optical hole 100, and there is no optical lens 4 on the avoidance hole 701, the optical lens 4 is cut out the optical hole 100. In the first and second use states of the binocular tube, the optical lens 4 is a single lens with negative optical power.

One end of the elastic sheet 71 is fixed, and the other end of the elastic sheet 71 forms a bent portion 710; the adjusting block 70 is provided with a first positioning groove 703 and a second positioning groove 704, and when the optical lens 4 is cut into the light hole 100, the bent portion 710 of the elastic sheet 71 is clipped into the first positioning groove 703 of the adjusting block 70; when the optical lens 4 is cut out the light hole 100, the bent portion 710 of the elastic sheet 71 is clipped into the second positioning groove 704 of the adjusting block 70, which can determine whether the optical lens 4 is switched in place by determining the elastic sheet 71 is clipped into the first positioning groove 703 or the second positioning groove 704.

The operating piece 72 is connected to the lower connecting base 10 and cooperates with the adjusting block 70 to control the up and down movement of the adjusting block 70, and if the operating piece 72 and the adjusting block 70 are connected by threads, the operating piece 72 can rotate relative to the lower connecting base 10 but cannot move, and the adjusting block 70 can move relative to the lower connecting base 10 but cannot rotate, in this way, by rotating the operating piece 72, the adjusting block 70 can be driven to move.

The movement guiding assembly comprises a guide holder 73 and a guide block 74, the guide holder 73 is connected to the lower connecting base 10, the guide block 74 is connected to the guide holder 73 and located on both sides of the adjusting block 70, a guide channel is formed between the guide holder 73 and the guide block 74, and extends in the direction of movement of the adjusting block 70, both ends of the adjusting block 70 are located inside the guide channel, which can ensure the stable movement of the adjusting block 70 through the movement guiding assembly.

In addition, in another implementation of the binocular tube in this embodiment, it has a third use state and a fourth use state, in the third use state, the optical lens 4 is cut out the optical hole 100, and the adjusting ring 60 moves to the first position setting in the direction away from the eyepiece tube holder 3; in the fourth use state, the optical lens 4 is cut into the optical hole 100, and the adjusting ring 60 moves to the second position setting in the direction towards the eyepiece tube holder 3. In the third and fourth use states of the binocular tube, the optical lens 4 is a single lens with positive optical power. The principles of the third and fourth use states are similar to those of the first and second use states, and will not be repeated here.

As shown in FIGS. 13 and 14, the binocular tube further comprises an optical lens assembly, the optical lens assembly comprises a first right-angle prism 80, a first lens group 81, an isosceles right-angle prism 82, a second right-angle prism 83, a Porro prism 84, a second lens group 85 and a third lens group 86 distributed in sequence along an optical path direction, and the first right-angle prism 80, the first lens group 81, the isosceles right-angle prism 82 and the second right-angle prism 83 are arranged within the connecting base assembly; the Porro prism 84 is arranged within the interpupillary distance adjusting base 2, and the second lens group 85 and the third lens group 86 are arranged within the eyepiece tube holder 3.
the focal length f_{G2} of the first lens group 81 satisfies 100 mm<f_{G2}<300 mm, the first lens group 81 comprises a single lens G2 with positive focal power and a doublet lens group G3 with negative focal power, and the doublet lens group G3 satisfies:

$$\frac{\vert {R}_{2} \vert}{{φ}_{2 / 2}} < 15 ,$$

where R₂ is the curvature radius of the cemented surface and φ₂ is the effective pore size of the cemented surface;
the focal length f_{G2} of the second lens group 85 satisfies −1<f_{G3}/f_{G2}<−0.2, the second lens group 85 comprises a doublet lens group G4 with negative focal power, and the doublet lens group G4 satisfies:

$$\frac{\vert {R}_{3} \vert}{{φ}_{3 / 2}} < 10 ,$$

where R₃ is the curvature radius of the cemented surface, and φ₃ is the effective pore size of the cemented surface;
the focal length f_{G4} of the third lens group 86 satisfies 1<f_{G2}/f_{G4}<10, the third lens group 86 comprises a doublet lens group with positive focal power, and there are no requirements for the curvature radius and effective pore size of its cemented surface.

The following are the parameters of the optical lens assembly when the binocular tube is in the first use state (Table 1) and the second use state (Table 2).

TABLE 1

| Optical lens | Face | | Thick- | | |
|---|---|---|---|---|---|
| assembly | No. | Radius | ness | Nd | Vd |
| Optical lens | 1 | −57 | 2 | 1.5891 | 61.2458 |
| | 2 | −62 | 3 | | |
| First right- | 3 | Infinity | 20 | 1.5168 | 64.2124 |
| angle prism | 4 | Infinity | 5 | | |
| First lens | 5 | 230 | 1.7 | 1.5891 | 61.2458 |
| group | 6 | −220 | 2.7 | | |
| | 7 | −46 | 1.4 | 1.6172 | 53.9284 |
| | 8 | 38 | 3.3 | 1.6131 | 60.6144 |
| | 9 | −48 | 5 | | |
| Isosceles right- | 10 | Infinity | 51 | 1.5168 | 64.2124 |
| angle prism | 11 | Infinity | 16 | | |
| Second right- | 12 | Infinity | 20 | 1.5168 | 64.2124 |
| angle prism | 13 | Infinity | 16 | | |
| Porro prism | 14 | Infinity | 93 | 1.5168 | 64.2124 |
| | 15 | Infinity | 5 | | |
| Second lens | 16 | 71 | 4.5 | 1.6172 | 53.9284 |
| group | 17 | −28 | 2 | 1.6204 | 60.3682 |
| | 18 | 35 | 3 | | |
| Third lens | 19 | 36 | 5 | 1.6172 | 53.9284 |
| group | 20 | −31 | 2 | 1.6131 | 60.6144 |
| | 21 | 103 | — | | |

TABLE 2

| Optical lens | Face | | Thick- | | |
|---|---|---|---|---|---|
| assembly | No. | Radius | ness | Nd | Vd |
| First right- | 1 | Infinity | 20 | 1.5168 | 64.2124 |
| angle prism | 2 | Infinity | 5 | | |
| First lens | 3 | 230 | 1.7 | 1.5891 | 61.2458 |
| group | 4 | −220 | 2.7 | | |
| | 5 | −46 | 1.4 | 1.6172 | 53.9284 |
| | 6 | 38 | 3.3 | 1.6131 | 60.6144 |
| | 7 | −48 | 5 | | |
| Isosceles right- | 8 | Infinity | 51 | 1.5168 | 64.2124 |
| angle prism | 9 | Infinity | 16 | | |
| Second right- | 10 | Infinity | 20 | 1.5168 | 64.2124 |
| angle prism | 11 | Infinity | 16 | | |
| Porro prism | 12 | Infinity | 93 | 1.5168 | 64.2124 |
| | 13 | Infinity | 5 | | |
| Second lens | 14 | 71 | 4.5 | 1.6172 | 53.9284 |
| group | 15 | −28 | 2 | 1.6204 | 60.3682 |
| | 16 | 35 | 3 | | |
| Third lens | 17 | 36 | 5 | 1.6172 | 53.9284 |
| group | 18 | −31 | 2 | 1.6131 | 60.6144 |
| | 19 | 103 | — | | |

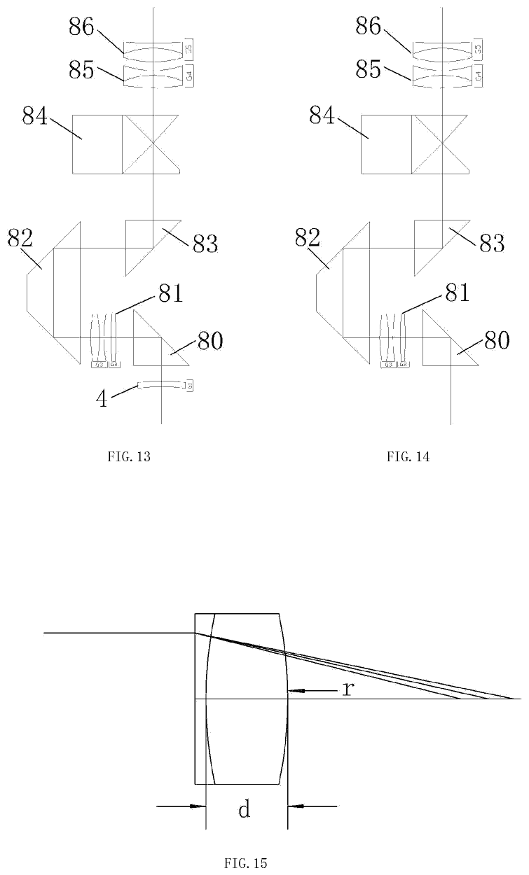

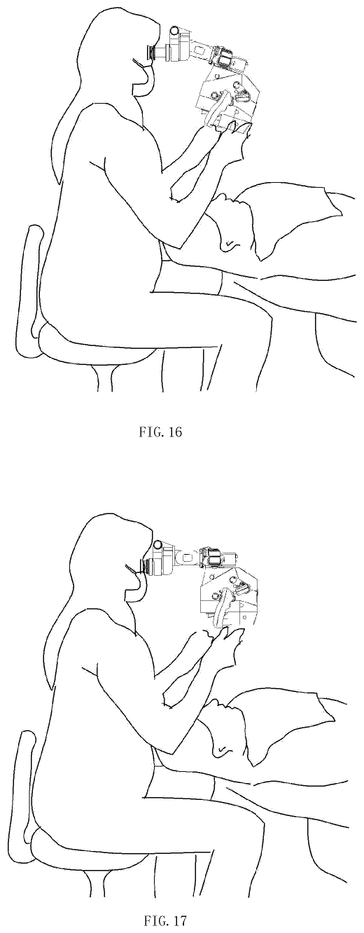

As shown in FIG. 15, and in Table 1 and Table 2,
The radius r is the curvature radius of the lens surface,
The thickness d is the center thickness of the lens,
Nd is the refractive index of d light (wavelength 589.3 nm) in an optical glass,
Explanation of Vd: Due to the difference in refractive index of the same transparent medium for light of different wavelengths, and the fact that white light is composed of various colored lights of different wavelengths, the special phenomenon of dispersion occurs when transparent materials refract white light, the Abbe number is an index used to represent the dispersion ability of a transparent medium, and the commonly used reference base is the central dispersion, which is the difference in refractive index between blue light and red light.

$$Abbe number Vd = ( nd - 1 ) / ( nF - nC ) ,$$

nd, nF, and nC are the refractive indices of D light, F light, and C light, respectively.
D light—yellow light, 589.3 nm, D line in sodium spectrum,
F light—blue light, 486.1 nm, F line in hydrogen spectrum,
C light—red light, 656.3 nm, C line in hydrogen spectrum.

The embodiments described above are only for illustrating the technical concepts and features of the present disclosure, and are intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1. An adjustable binocular tube, comprising a connecting base assembly, an interpupillary distance adjusting base, an eyepiece tube holder, and an eyepiece tube assembly, the interpupillary distance adjusting base being connected to the connecting base assembly, the eyepiece tube holder being connected to the interpupillary distance adjusting base, the eyepiece tube assembly being connected to the eyepiece tube holder, and an optical hole being formed in the connecting base assembly, wherein
the eyepiece tube assembly is connected to the eyepiece tube holder by means of an adjusting mechanism, the adjusting mechanism comprises an adjusting ring, the adjusting ring is connected to the eyepiece tube holder, and the adjusting ring is movable relative to the eyepiece tube holder in an axial direction of the adjusting ring, and the eyepiece tube assembly is connected to the adjusting ring; the connecting base assembly is provided with an optical lens capable of cutting into or cutting out the optical hole.

2. The adjustable binocular tube according to claim 1, wherein the binocular tube has a first use state and a second use state, and in the first use state, the adjusting ring moves to a first position setting in a direction away from the eyepiece tube holder, and the optical lens is cut into the optical hole; in the second use state, the adjusting ring moves to a second position setting in a direction towards the eyepiece tube holder, and the optical lens is cut out the optical hole;
or, the binocular tube has a third use state and a fourth use state, in the third use state, the optical lens is cut out the optical hole, and the adjusting ring moves to a first position setting in a direction away from the eyepiece tube holder; in the fourth use state, the optical lens is cut into the optical hole, and the adjusting ring moves to a second position setting in a direction towards the eyepiece tube holder.

3. The adjustable binocular tube according to claim 2, wherein one end of the adjusting ring is inserted into the interior of the eyepiece tube holder from one end of the eyepiece tube holder, and the adjusting ring is movable to extend out of the eyepiece tube holder or retract into the eyepiece tube holder; the eyepiece tube assembly is connected to the other end of the adjusting ring.

4. The adjustable binocular tube according to claim 3, wherein the eyepiece tube assembly is inserted into the other end of the adjusting ring, and a sleeve body is provided between the eyepiece tube assembly and the adjusting ring.

5. The adjustable binocular tube according to claim 3, wherein the adjusting mechanism further comprises a first limit ring connected to one end of the eyepiece tube holder, and when in the first position setting, the adjusting ring moves to extend from the eyepiece tube holder until one end of the adjusting ring is in contact with the first limit ring.

6. The adjustable binocular tube according to claim 5, wherein the adjusting mechanism further comprises a second limit ring fixedly sleeved on the other end of the adjusting ring, and when in the second position setting, the adjusting ring moves to retract into the eyepiece tube holder until the second limit ring is in contact with the first limit ring.

7. The adjustable binocular tube according to claim 1, wherein the adjusting ring is threadedly connected to the eyepiece tube holder and/or, wherein one of the adjusting ring and the eyepiece tube holder is provided with a sliding groove, and the other is provided with a sliding block, the sliding groove extends in the axial direction of the adjusting ring, and the sliding block is located inside the sliding groove.

8. (canceled)

9. The adjustable binocular tube according to claim 1, wherein the optical lens is arranged on the connecting base assembly via a switching mechanism, the switching mechanism comprises an adjusting block flexibly connected to the connecting base assembly and provided with a mounting hole and an avoidance hole, the optical lens is arranged on the mounting hole, and when the mounting hole is coaxial with the optical hole, the optical lens is cut into the optical hole; when the avoidance hole is coaxial with the optical hole, the optical lens is cut out the optical hole.

10. The adjustable binocular tube according to claim 9, wherein the switching mechanism further comprises an elastic sheet, one end of the elastic sheet is fixed, and the other end forms a bent portion; the adjusting block is provided with a first positioning groove and a second positioning groove, when the optical lens is cut into the optical hole, the bent portion of the elastic sheet is clipped into the first positioning groove of the adjusting block; when the optical lens is cut out the optical hole, the bent portion of the elastic sheet is clipped into the second positioning groove of the adjusting block, which can determine whether the optical lens is switched in place by determining the elastic sheet is clipped into the first or second positioning groove.

11. The adjustable binocular tube according to claim 9, wherein the adjusting block is movably connected to the connecting base assembly.

12. The adjustable binocular tube according to claim 11, wherein the switching mechanism further comprises an operating piece connected to the connecting base assembly and cooperating with the adjusting block to control the movement of the adjusting block;
the operating piece is threadedly connected to the adjusting block.

13. The adjustable binocular tube according to claim 11, wherein the switching mechanism further comprises a movement guiding assembly comprising a guide holder and a guide block, the guide holder is connected to the connecting base assembly, the guide block is connected to the guide holder and located on at least one side of the adjusting block, a guide channel is formed between the guide holder and the guide block and extends in the direction of movement of the adjusting block, the adjusting block is located within the guide channel.

14. The adjustable binocular tube according to claim 11, wherein the adjusting block is movable in an up-down direction relative to the connecting base assembly, and the mounting hole and the avoidance hole are arranged in the up-down direction.

15. The adjustable binocular tube according to claim 1, wherein the optical lens is a single lens with negative optical power or a single lens with positive optical power.

16. The adjustable binocular tube according to claim 1, wherein the binocular tube further comprises an optical lens assembly, the optical lens assembly comprises a first right-angle prism, a first lens group, an isosceles right-angle prism, a second right-angle prism, a Porro prism, a second lens group and a third lens group distributed in sequence along the optical path direction.

17. The adjustable binocular tube according to claim 16, wherein the focal length f_{G2} of the first lens group satisfies 100 mm<f_{G2}<300 mm,
the focal length f_{G3} of the second lens group satisfies −1<f_{G3}/f_{G2}<−0.2,
the focal length f_{G4} of the third lens group satisfies 1<f_{G2}/f_{G4}<10.

18. The adjustable binocular tube according to claim 17, wherein the first lens group comprises a single lens with positive focal power and a doublet lens group with negative focal power, and the doublet lens group satisfies:
$$\frac{\vert {R}_{2} \vert}{\frac{{φ}_{2}}{2}} < 15 ,$$
where R₂ is the curvature radius of the cemented surface, and φ₂ is the effective pore size of the cemented surface;
the second lens group comprises a doublet lens group with negative focal power, and the doublet lens group satisfies:
$$\frac{\vert {R}_{3} \vert}{\frac{{φ}_{3}}{2}} < 10 ,$$
where R₃ is the curvature radius of the cemented surface, and φ₃ is the effective pore size of the cemented surface;
the third lens group comprises a doublet lens group with positive focal power.

19. The adjustable binocular tube according to claim 16, wherein the first right-angle prism, the first lens group, the isosceles right-angle prism and the second right-angle prism are arranged within the connecting base assembly; the Porro prism is arranged within the interpupillary distance adjusting base, and the second lens group and the third lens group are arranged within the eyepiece tube holder.

20. The adjustable binocular tube according to claim 1, wherein the connecting base assembly comprises a lower connecting base and an upper connecting base, the lower connecting base and the upper connecting base are rotatably connected to each other, the optical lens is arranged on the lower connecting base, and the interpupillary distance adjusting base is connected to the upper connecting base and/or, wherein the eyepiece tube assembly comprises an eyepiece tube, an eyepiece adjusting tube, a diopter ring, a diopter adjusting ring and an eyepiece diaphragm, the eyepiece tube is connected to the adjusting ring, the eyepiece adjusting tube is connected to the eyepiece tube, the diopter ring is connected to the eyepiece adjusting tube through the diopter adjusting ring, and the eyepiece diaphragm is connected to the eyepiece tube.

21. (canceled)

22. A surgical microscope, wherein it comprises the adjustable binocular tube according to claim 1.
